(19) United States
(12) Patent Application Publication
(10) Pub. No.: US 2010/0166687 A1
(43) Pub. Date: Jul. 1, 2010

(54) COSMETIC LIGHT PROTECTION AGENT

(75) Inventors: Karin Golz-Berner, Monaco (MC); Sandra Miton, Nice (FR); Leonhard Zastrow, Monaco (MC)

Correspondence Address: Gregory A. Nelson;Novak Druce & Quigg LLP, 525 Okeechobee Blvd, Suite 1500, West Palm Beach, FL, 33401, US

(73) Assignee: COTY PRESTIGE LANCASTER GROUP GMBH, Mainz (DE)

(21) Appl. No.: 12/529,324

(22) PCT Filed: Feb. 29, 2008
(86) PCT No.: PCT/EP08/52507

§ 371 Date: Feb. 12, 2010

(30) Foreign Application Priority Data
Mar. 1, 2007 (DE) 10 2007 010 861.5

(51) Int. Cl. A61K 8/49; A61Q 17/04; A61K 8/37

(52) U.S. Cl. 424/60; 424/59

(57) ABSTRACT

The invention relates to a cosmetic light-protective agent for wavelengths of from 250 to 1400 nm, containing at least methylene bis-benzotriazolyl tetramethylbutylphenol as UVA filter; at least one UVB filter; sea buckthorn oil with a refractive index of from 1.4 to 1.59 and a solid with a d₅₀ of from 3 to 50 μm, selected among citrine, topaz, amethyst, ruby, glass with a refractive index of from 1.500 to 1.599 and mixtures thereof; at least one infrared-absorbing glass with a d₅₀ of from 5 to 50 μm; a free-radical scavenger, and cosmetic adjuvants and/or excipients and optionally further cosmetic active substances. The agent shows an unexpectedly high light absorption and largely prevents skin damage caused by light of 250-1400 nm in wavelength.

The invention relates to a cosmetic light-protective agent having effect for a large wavelength range. The agent has an unexpectedly high light absorption and largely prevents skin damage caused by light of 250-1400 nm wavelengths.

A large number of sunscreens having various UVA and UVB filters are known. Also, combinations of UVA, UVB and IR filters are known from WO 2006/136724 wherein monodisperse silicon dioxide or polystyrene as well as acrylic polymers are combined in addition to conventional UV filters. WO 03/045345 describes cosmetic compositions including various white and colored glasses in order to improve the UV and IR behavior and skin feeling.

The invention is based on the object of developing a cosmetic light-protective agent which develops special light-protective properties in a wide range of the spectrum of light, thereby allowing universal use.

According to the invention, the light-protective agent is characterized in that it covers the wavelength range of from 250 nm to 1400 nm and comprises at least the following ingredients:
(a) at least methylene bis-benzotriazolyl tetramethylbutylphenol as UVA filter;
(b) at least one filter substance for UVB radiation;
(c) sea buckthorn oil with a refractive index of from 1.4 to 1.59 and a solid with a particle size d₅₀ of from 3 to 50 μm, selected among citrine, topaz, amethyst, ruby, glass with a refractive index of from 1.500 to 1.599 at 587.6 nm or mixtures thereof;
(d) at least one infrared-absorbing glass with a particle size d₅₀ of from 5 to 50 μm;
(e) a free-radical scavenging substance or mixture of substances;

the total share of light-protective substances (a) through (d) ranging from 9 to 32 wt. %, and the balance to make 100%, apart from ingredient (e), being cosmetic adjuvants and/or excipients and optionally further cosmetic active substances.

From a wavelength range of 250-400 nm of the UV region via the range of visible light of >400-700 nm and up to the infrared range of >700 nm to 1400 nm, the mixture according to the invention was found to have unexpectedly high absorption of light, thus being capable of largely preventing skin damage caused by light of all these wavelengths. Surprisingly, the effect to be expected from the single ingredients of the mixture according to the invention is significantly surpassed by the combination of all ingredients, thus representing true synergism.

Particularly preferred as ingredient (c) is a mixture of an optical clear glass, preferably with a particle size d₅₀ of from 3 to 30 μm, and ground ruby or citrine, preferably with a particle size d₅₀ of from 5 to 40 μm. The optical glass can be colored glass, e.g. red glass. In addition to sea buckthorn oil, the ingredient (c) can include ground ruby or citrine only.

The expression “particle size d₅₀” is understood to be a particle size distribution with a Gaussian distribution curve wherein at least 50% of the particles show the size as indicated by the particle size value.

A glass, preferably a green glass, such as Schott No. 8516 which has a specific transmission factor (λ=1100 nm; d=0.5 mm) of 16 and a particle size d₅₀ of 12 μm and d₉₉ of 63 μm, from the glass system SiO₂—Al₂O₃—BaO—Fe₂O₃ including shares of Sb₂O₃ and B₂O₃ as well as Li₂O, is used as filter for infrared radiation. However, other IR glasses can also be used. The share of IR glass can be in the range of from 0.2 to 1.6 wt. % with advantage.

The share of methylene bis-benzotriazolyl tetramethylbutylphenol (a) is preferably in the range of 3 to 8 wt. %, especially 4 to 6 wt. %, relative to the overall weight of the light-protective agent.

The share of filter substance for UVB radiation (b) is preferably in the range of from 4 to 20 wt. %, especially 5 to 18 wt. %. It is preferably a mixture of several filter substances. The filter substance for UVB radiation is selected e.g. from the group comprising isoamyl p-methoxycinnamate, ethylhexyl triazone, ethylhexyl methoxycinnamate, octocrylene, phenylbenzimidazole sulfonic acid, diethylhexyl butamido triazone, ethylhexyl salicylate and mixtures thereof.

The share of sea buckthorn oil is in the range of from 0.1 to 1.5 wt. %. The sea buckthorn oil employed herein is a sea buckthorn pulp oil obtained from non-dried berries by the action of native enzymes and centrifuging the oil at a temperature of 1-50° C. The sea buckthorn oil, with a weight percentage in the light-protective agent as specified, is not a substitute for other fats and oils which form the actual oil phase, if present, and will be described below.

The total amount of solid, which can also be a mixture of various solids, is advantageously in the range of from 0.5 to 4 wt. %, especially 0.8 to 2.5 wt. %. Particularly preferred is a combination of 0.1 to 0.8 wt. % ruby powder and 0.8 to 1.8 wt. % optical glass of the system SiO₂—Na₂O—CaO—ZnO—BaO which may include CuO, ZnO and MgO and is advantageously a red glass, e.g. Schott D 8010.

Well-known antioxidants can be used as free-radical scavengers, including vitamins such as vitamin C and derivatives thereof, e.g. ascorbyl acetate, phosphate and palmitate, magnesium ascorbyl phosphate; vitamin A and derivatives thereof; folic acid and derivatives thereof, vitamin E and derivatives thereof, such as tocopheryl acetate; flavones or flavonoids; amino acids such as histidine, glycine, tyrosine, tryptophan and derivatives thereof; imidazoles such as cis- or trans-urocaninic acid and derivatives thereof; peptides such as D,L-carnosine, D-carnosine, L-carnosine and derivatives thereof; carotenoids and carotenes such as α-carotene and β-carotene, lycopene; uric acid and derivatives thereof; α-hydroxy acids such as citric acid, lactic acid, malic acid; α-hydroxyfatty acids such as palmitic acid, phytic acid; plant extracts and mixtures thereof, such as extracts of pine bark, rosemary, oregano, calendula, quebracho, angelica roots, camellia, green coffee extract, green tea extract etc., as well as yeast extracts.

An especially advantageous free-radical scavenger is a preparation having a content of a product obtained by extraction of the bark of Quebracho blanco and subsequent enzymatic hydrolysis, which product includes at least 90 wt. % of proanthocyanidin oligomers and at most 10 wt. % of gallic acid, in microcapsules, and a silkworm extract obtained by extraction, which includes the peptide cecropin, amino acids and a vitamin mixture, and a non-ionic, cationic or anionic hydrogel or mixture of hydrogels, and one or more phospholipids, and water (e.g. WO 99/66881, Example 1).

Another advantageous free-radical scavenger is a mixture of alcohol-based plant extracts free of liposomes, consisting of an extract of green coffee beans, an extract of Camellia sinensis leaves, of Pongamia pinnata, and of Angelica archangelica roots and a monohydric lower alcohol (INCI: RPF complex II, or WO 01/26617, Example 1).

An advantageous light-protective agent according to the invention comprises the following ingredients:
(a) at least methylene bis-benzotriazolyl tetramethylbutylphenol as UVA filter;
(b) a filter substance for UVB radiation selected among isoamyl p-methoxycinnamate, ethylhexyl triazone, ethylhexyl methoxycinnamate, octocrylene, phenylbenzimidazole sulfonic acid, diethylhexyl butamido triazone, ethylhexyl salicylate and mixtures thereof;
(c) a mixture of sea buckthorn oil and ground ruby or citrine;
(d) an IR glass with a particle size d₅₀ of from 5 to 50 μm;
(e) a free-radical scavenging preparation having a content of a product obtained by extraction of the bark of Quebracho blanco and subsequent enzymatic hydrolysis, which product includes at least 90 wt. % of proanthocyanidin oligomers and at most 10 wt. % of gallic acid, in microcapsules, and a silkworm extract obtained by extraction, which includes the peptide cecropin, amino acids and a vitamin mixture, and a non-ionic, cationic or anionic hydrogel or mixture of hydrogels, and one or more phospholipids, and water.

In addition, the light-protective agent according to the invention includes cosmetic adjuvants and/or excipients as usually employed in such preparations, e.g. water, preservatives, dyes, pigments with a coloring effect, thickeners, fragrances, alcohols, polyols, esters, electrolytes, gelling agents, polar and non-polar oils, polymers, copolymers, emulsifiers, waxes, stabilizers, penetration enhancers.

The light-protective agent may also include one or more additional cosmetic active substances, including e.g. self-tanning agents, moisturizers, melanin, antiph-logistic natural active substances, oxygen-loaded asymmetric lamellar aggregates according to WO 94/00109, as well as those for positively influencing aging skin, especially in combination with bioquinones, particularly ubiquinone Q10, creatine, creatinine, carnitine, biotin, isoflavones, cardiolipin, lipoic acid, antifreeze proteins, arctiin, hop extracts and hop-malt extracts.

In a preferred embodiment of the invention the light-protective agent includes melanin. Sea buckthorn oil and melanin are included at a ratio of 1:0.01 to 0.1, and the maximum amount of soluble melanin can be around 0.1 wt. %, preferably 0.03 wt. %, relative to the overall weight of the light-protective agent.

Also, active substances for supporting the skin functions in cases of dry skin can be added, such as, in particular, vitamin C, biotin, carnitine, creatine, propionic acid, green tea extracts, eucalyptus oil, urea, and mineral salts, particularly NaCl, marine minerals, as well as osmolytes.

Active substances for alleviating and/or positively influencing irritative skin conditions can also be used, particularly sericosides, extracts of licorice, licochalcones, particularly licochalcone A, silymarin, silyphos, dexpanthenol.

Active substances which have an influence on pigmentation and can be additionally included in the light-protective agents of the invention are in particular tyrosine sulfate, 8-hexadecene-1,16-dicarboxylic acid, lipoic acid, liponamide, extracts of licorice, kojic acid, hydroquinone, arbutin, fruit acids, particularly α-hydroxy acids (AHAs), Uvae ursi, ursolic acid, ascorbic acid, green tea extracts, aminoguanidine, pyridoxamine. Also included are substances causing enhanced or more rapid tanning of the skin, particularly lipofuscins, purines and pyrimidines, dihydroxyacetone, erythrulose.

In any event, the light-protective agents according to the invention include at least one additional UVB filter. However, it is also possible to add several water-soluble and/or oil-soluble UVA or UVB filters, or both. The so-called broad-spectrum filters are also “UVB filters” in the meaning of the invention.

Convenient oil-soluble UVB filters include 4-aminobenzoic acid derivatives such as 2-ethylhexyl 4-dimethylaminobenzoate; esters of cinnamic acid such as 2-ethylhexyl 4-methoxycinnamate; benzophenone derivatives such as 2-hydroxy-4-methoxybenzophenone; 3-benzylidenecamphor derivatives such as 3-benzylidenecamphor.

Preferred oil-soluble UV filters are benzophenone-3, butyl-methoxybenzoylmethane, octyl methoxycinnamate, octyl salicylate, 4-methylbenzylidenecamphor, homosalate and octyl dimethyl PABA.

Water-soluble UVB filters are, for instance, sulfonic acid derivatives of benzophenone or of 3-benzylidenecamphor or salts such as the Na or K salt of 2-phenylbenzimidazole-5-sulfonic acid.

UVA filters which can be used together with methylene bis-benzotriazolyl tetramethylbutylphenol include benzophenone-3, dibenzoylmethane derivatives such as 1-phenyl-4-(4′-isopropylphenyl)propane-1,3-dione, butyl methoxybenzoylmethane or menthyl anthranilate, as well as disodium phenyl dibenzylimadazole tetrasulfonate and diethylamino hydroxybenzoyl hexylbenzoate.

Particularly preferred are benzophenone-3, butylmethoxydibenzoylmethane, octyl methoxycinnamate, octyl salicylate, 4-methylbenzylidenecamphor, homosalate, octocrylene, ethylhexyl methoxycinnamate, isoamyl p-methoxycinnamate, octyl dimethyl PABA, ethylhexyltriazone, diethylhexyl butamido triazone, ethylhexyl salicylate, disodium phenyl dibenzimidazole tetrasulfonate, bis-ethylhexyloxyphenol methoxyphenyl triazine.

Furthermore, broad-spectrum filters such as bis-resorcinyltriazine derivatives or benzoxazoles can be employed.

Also usable as sunscreen filters are inorganic pigments based on metal oxides, such as TiO₂, SiO₂, ZnO, Fe₂O₃, ZrO₂, MnO, Al₂O₃, which can also be used in a mixture.

Particularly preferred as inorganic filters are agglomerated substrates of TiO₂ and/or ZnO, which have a content of spherical and porous SiO₂ particles, said SiO₂ particles having a particle size in the range of 0.05 μm to 1.5 μm and, along with the SiO₂ particles, other inorganic particulate substances with a spherical structure, said spherical SiO₂ particles with the other inorganic substances forming defined agglomerates with a particle size in the range of 0.06 μm to 5 μm (according to WO 99/06012).

Especially suitable oils for the light-protective agents according to the invention are, for example, silicone oils, mineral oils, hydrogenated polyisobutene, polyisoprene, squalanes, tridecyl trimellitate, trimethylpropane triisostearate, isodecyl citrate, neopentyl glycol diheptanoate, PPG-15 stearyl ether as well as vegetable oils, such as calendula oil, jojoba oil, avocado oil, macadamia nut oil, castor oil, cocoa butter, coconut oil, maize oil, cottonseed oil, olive oil, palm nut oil, rapeseed oil, safflower oil, sesame oil, soyabean oil, sunflower seed oil, wheat germ oil, grapeseed oil, candlenut oil and mixtures thereof, as well as cosmetic esters or ethers, which may be branched or unbranched, saturated or unsaturated, or mixtures of two or more of these oils, esters or ethers.

The agent according to the invention may also include customary humectants such as glycerol, butylene glycol, propylene glycol and mixtures thereof.

The light-protective agent according to the invention can be prepared in the form of an emulsion or gel so that gelling agents suitable for gels can be used, including e.g. carbomer, xanthan gum, carrageenan, acacia gum, guar gum, agar, alginates and tyloses, carboxymethylcellulose, hydroxyethylcellulose, quaternized cellulose, quaternized guar, specific polyacrylates, polyvinyl alcohol, polyvinyl-pyrrolidone, montmorillonite.

Suitable emulsifiers for O/W emulsions are e.g. addition products of 2-30 moles of ethylene oxide with linear C₈-C₂₂ fatty alcohols, with C₁₂-C₂₂ fatty acids and with C₈-C₁₅ alkylphenols; C₁₂-C₂₂ fatty acid mono- and diesters of addition products of 1-30 moles of ethylene oxide with glycerol.

Suitable emulsifiers for W/O emulsions are, for example, addition products of 2-15 moles of ethylene oxide with castor oil; esters of C₁₂-C₂₂ fatty acids and glycerol, polyglycerol, pentaerythritol, sugar alcohols (e.g. sorbitol), polyglucosides (e.g. cellulose); polyalkylene glycols; wool wax alcohols; copolymers of polysiloxane-polyalkyl polyethers.

Addition of electrolytes causes a change in the solubility of a hydrophilic emulsifier. Hydrophilic emulsifiers undergo partial phase inversion during which the oil phase solubilizes water. The result is a stable emulsion, in particular a micro-emulsion or an O/W/O-emulsion. Suitable electrolytes are salts including the following anions: chlorides, inorganic oxo-element anions, such as borates, aluminates, sulfates, phosphates, carbonates. Electrolytes based on organic anions include citrates, tartrates, lactates, propionates, acetates and benzoates as well as EDTA and salts thereof.

Cations of the salts can be alkali metal ions, alkaline earth metal ions, ammonium ions, alkyl ammonium ions, iron ions, zinc ions.

The light-protective agent according to the invention can be used e.g. in sun creams, sun gels, sun sprays, aerosols, after-sun products, day creams, night creams, masks, body lotions, make-ups, lip sticks, lip balm, eye beauty care, hair masks, hair shampoos, shower gels, shower oils, bath oils. The production of such products is effected in a way well-known to a person skilled in this field.

The invention is also directed to a light-protective agent which covers the wavelength range of from 400 to 700 nm and comprises sea buckthorn oil with a refractive index of from 1.4 to 1.59 and a solid with a particle size d₅₀ of from 3 to 50 μm, selected among citrine, topaz, amethyst, ruby, glass with a refractive index of from 1.500 to 1.599 at 587.6 nm or mixtures thereof, and a free-radical scavenging substance or mixture of substances, and cosmetic adjuvants and/or excipients and optionally further cosmetic active substances.

The invention also relates to the use of a mixture of the following ingredients:
(a) at least methylene bis-benzotriazolyl tetramethylbutylphenol as UVA filter;
(b) at least one filter substance for UVB radiation;
(c) sea buckthorn oil with a refractive index of from 1.4 to 1.59 and a solid with a particle size d₅₀ of from 3 to 50 μm, selected among citrine, topaz, amethyst, ruby, glass with a refractive index of from 1.500 to 1.599 at 587.6 nm or mixtures thereof;
(d) at least one infrared-absorbing glass with a particle size d₅₀ of from 5 to 50 μm;
(e) a free-radical scavenging substance or mixture of substances;

the total share of light-protective substances (a) through (d) ranging from 9 to 32 wt. %, and the balance to make 100%, apart from ingredient (e), being cosmetic adjuvants and/or excipients and optionally further cosmetic active substances;

as a cosmetic light-protective agent in the wavelength range of from 250 to 1400 nm.

With reference to the examples, the invention will be illustrated in more detail below. All data are given in percent by weight, unless otherwise stated.

### EXAMPLE 1

### Light-Protective Cream 1

Phase A

| Water | q.s. ad 100 |
|---|---|
| Xanthan gum | 0.25 |
| Propylene glycol | 2 |
| Acrylates/vinyl isodecanoate crosspolymer | 0.3 |
| Cyclopentasiloxane & cyclohexasiloxane & | |
| cyclotetrasiloxane | 3 |
| Tetrasodium EDTA | 0.1 |
| Sodium hydroxide | 0.1 |

Phase B

| | |
|---|---|
| Isoamyl p-methoxycinnamate | 5 |
| Octocrylene | 3 |
| Butyl methoxydibenzoylmethane | 4 |
| Bis-ethylhexyloxyphenol methoxyphenyl triazine | 1 |
| Methylene bis-benzotriazolyl tetramethylbutylphenol | 5 |
| Dicaprylyl carbonate & tocopherol | 6 |

Phase C

| | |
|---|---|
| RPF complex I¹ | 1 |
| Hippophae rhamnoides | 0.5 |
| Glass (Schott D8010) | 3.0 |
| Ruby powder | 0.1 |
| Glass (Schott 8516) | 1.5 |
| Ethanol | 3 |
| Preservative | 0.9 |
| Perfume oil | 0.75 |

¹In accordance with WO 99/66881, active substance complex of Example 1

The phases A and B are produced separately, combined at 75° C. and mixed carefully with stirring. After cooling the mixture, phase C is added at 45° C. with stirring.

### EXAMPLE 2

### Light-Protective Cream 2

Phase A

| Water | q.s. ad 100 |
|---|---|
| Xanthan gum | 0.25 |
| Propylene glycol | 2 |
| Acrylates/vinyl isodecanoate crosspolymer | 0.3 |
| Cyclopentasiloxane & cyclohexasiloxane & | |
| cyclotetrasiloxane | 3-5 |
| Tetrasodium EDTA | 0.1 |
| Sodium hydroxide | 0.1 |

Phase B

| | |
|---|---|
| Isoamyl p-methoxycinnamate | 4-5 |
| Octocrylene | 2 |
| Butyl methoxydibenzoylmethane | 3 |
| Bis-ethylhexyloxyphenol methoxyphenyl triazine | 1 |
| Methylene bis-benzotriazolyl tetramethylbutylphenol | 4 |
| Dicaprylyl carbonate & tocopherol | 5 |

Phase C

| | |
|---|---|
| RPF complex I¹ | 0.5 |
| Hippophae rhamnoides | 0.3 |
| Glass (Schott D8010) | 1.0 |
| Ruby powder | 0.2 |
| Glass (Schott 8516) | 0.5 |
| Melanin, soluble | 0.01 |
| Ethanol | 3 |
| Preservative | 0.9 |
| Perfume oil | 0.75 |

¹In accordance with WO 99/66881, active substance complex of Example 1

The production is as in Example 1.

### Example 3

### Comparative Example

It is well-known that UVB radiation causes formation of free radicals in the skin, which, given sufficient time of exposure to radiation, give rise to dermal erythemas. Among other things, this forms the basis of specifying the so-called LSF (“Lichtschutzfaktor”) or SPF (sun protection factor) labelled on cosmetic sunscreen products.

Furthermore, it is well-known that the number of free radicals in the skin is massively increased and the effect of free radicals in the skin is increased when considering the UVA radiation in addition to the UVB radiation so that a so-called integral sun protection factor (ISPF) has been suggested (WO 2005/103659).

It has now been found that radiation in the visible region as well as infrared radiation can also give rise to free radicals in the skin so that expansion of the integral sun protection factor over the entire range of wavelengths of from 250 to 1400 nm would be reasonable.

Setting out from this conception, which requires a number of further investigations, preliminary measurements of free radicals using the methodology specified in WO 2005/103659 (skin biopsies) were effected in different wavelength ranges and, using the active substances, expressed as ISPF.

Taken as a group for a particular wavelength range, the active substances of Example 1 show the following results:

18% UVA/UVB filter¹ for the range of 200-400 nm=ISPF_{UVA/B}=7.6 ¹Isoamyl p-methoxycinnamate, octocrylene, butyl methoxydibenzoylmethane, bis-ethylhexyloxyphenol methoxyphenyl triazine, methylene bis-benzotriazolyl tetramethylbutylphenol

1.5% filter² for the range of 400-700 nm=ISPF_{VIS}=2.0 ²Hippophae rhamnoides, glass (Schott D6010), ruby powder

0.5% filter³ for the range of 700-1400 nm=ISPF_{IR}=1.3 Glass (Schott 8516)

From the above, a theoretical total ISPF of 7.6+2+1.3=10.9 would be expected.

However, a practical total ISPF of 13.9 was found when investigating the ISPF for the overall mixture of all filters.

This shows an unexpectedly high effect of the mixture of all active substances compared to the individual groups, which cannot be derived from a combination of the individual ingredients and their specifically known effectiveness. This synergism highlights the significance of the invention per se and the significance of covering all free radicals in the above-mentioned wavelength range and taking them into account when providing new light-protective agents.

We claim:

1. A light-protective agent that protects against electromagnetic radiation having a wavelength range of from 250 nm to 1400 nm, comprising:
(a) at least methylene bis-benzotriazolyl tetramethylbutylphenol as UVA filter;
(b) at least one filter substance for UVB radiation;
(c) sea buckthorn oil with a refractive index of from 1.4 to 1.59 and a solid with a particle size d₅₀ of from 3 to 50 μm, selected among citrine, topaz, amethyst, ruby, glass with a refractive index of from 1.500 to 1.599 at 587.6 nm or mixtures thereof;
(d) at least one infrared-absorbing glass with a particle size d₅₀ of from 5 to 50 μm;
(e) a free-radical scavenging substance or mixture of substances;
the total share of light-protective substances (a) through (d) ranging from 9 to 32 wt. %, and the balance to make 100%, apart from ingredient (e), being cosmetic adjuvants and/or excipients and optionally further cosmetic active substances.

2. The light-protective agent according to claim 1, wherein said further cosmetic active substances comprise melanin.

3. The light-protective agent according to claim 1, wherein substance (c) comprises a mixture of sea buckthorn oil and a ground clear glass with a refractive index of from 1.50 to 1.59 and a ground ruby.

4. The light-protective agent according to claim 1 comprising:
(a) at least methylene bis-benzotriazolyl tetramethylbutylphenol as UVA filter;
(b) a filter substance for UVB radiation selected among isoamyl p-methoxycinnamate, ethylhexyl triazone, ethylhexyl methoxycinnamate, octocrylene, phenylbenzimidazole sulfonic acid, diethylhexyl butamido triazone, ethylhexyl salicylate and mixtures thereof;
(c) a mixture of sea buckthorn oil and ground ruby or citrine;
(d) an IR glass with a particle size d₅₀ of from 5 to 50 μm;
(e) a free-radical scavenging preparation having a content of a product obtained by extraction of the bark of Quebracho blanco and subsequent enzymatic hydrolysis, which product includes at least 90 wt. % of proanthocyanidin oligomers and at most 10 wt. % of gallic acid, in microcapsules, and a silkworm extract obtained by extraction, which includes the peptide cecropin, amino acids and a vitamin mixture, and a non-ionic, cationic or anionic hydrogel or mixture of hydrogels, and one or more phospholipids, and water.

5. A light-protective agent, that it protects against electromagnetic radiation having a wavelength range of from 400 to 700 nm comprising:
sea buckthorn oil with a refractive index of from 1.4 to 1.59 and a solid with a particle size d₅₀ of from 3 to 50 μm, selected among citrine, topaz, amethyst, ruby, glass with a refractive index of from 1.500 to 1.599 at 587.6 nm or mixtures thereof; and
a free-radical scavenging substance or mixture of substances, and cosmetic adjuvants, excipients and/or further cosmetic active substances.

6. A method of protecting skin against electromagnetic radiation having a wavelength range of from 250 nm to 1400 nm comprising:
applying a cosmetic light-protective agent to skin, said light-protective agent comprising:
(a) at least methylene bis-benzotriazolyl tetramethylbutylphenol as UVA filter;
(b) at least one filter substance for UVB radiation;
(c) sea buckthorn oil with a refractive index of from 1.4 to 1.59 and a solid with a particle size d₅₀ of from 3 to 50 μm, selected among citrine, topaz, amethyst, ruby, glass with a refractive index of from 1.500 to 1.599 at 587.6 nm or mixtures thereof;
(d) at least one infrared-absorbing glass with a particle size d₅₀ of from 5 to 50 μm;
(e) a free-radical scavenging substance or mixture of substances;
the total share of light-protective substances (a) through (d) ranging from 9 to 32 wt. %, and the balance to make 100%, apart from ingredient (e), being cosmetic adjuvants and/or excipients and optionally further cosmetic active substances.
